(19) United States
(12) Patent Application Publication
(10) Pub. No.: US 2005/0227268 A1
(43) Pub. Date: Oct. 13, 2005

(54) Expression cloning using a tagged cDNA library

(75) Inventors: Leonard I. Zon, Wellesley, MA (US); Sadhana Agarwal, Cambridge, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Children's Medical Center Corporation, Boston, MA (US)

(21) Appl. No.: 11/051,938

(22) Filed: Feb. 4, 2005

Related U.S. Application Data
(63) Continuation of application No. 10/051,452, filed on Jan. 18, 2002, now abandoned.
Application No. 10/051,452 is a continuation of application No. PCT/US00/19960, filed on Jul. 20, 2000.
(60) Provisional application No. 60/145,044, filed on Jul. 22, 1999.

(51) Int. Cl. C12Q 1/68; C12N 15/74

(52) U.S. Cl. 435/6; 435/471

(57) ABSTRACT

Methods of expression cloning where a cDNA construct expresses a tagged polypeptide for a biochemical activity of interest are described.

### RELATED APPLICATIONS

This application is a continuation of U.S. application Ser. No. 10/051,452, filed Jan. 18, 2002 which is a continuation of International Application No. PCT/US00/19966, which designated the United States and was filed Jul. 22, 2000, published in English, which claims the benefit of U.S. Provisional Application No. 60/145,044, filed Jul. 22, 1999.

The entire teachings of the above applications are incorporated herein by reference.

### GOVERNMENT SUPPORT

The invention was supported, in whole or in part, by a National Institutes of Health grant No. 2 po1 h132262-15. The Government has certain rights in the invention.

### BACKGROUND OF THE INVENTION

Complex processes such as cell growth and differentiation are tightly controlled in normal cells. Loss of this control leads to several diseased states including various forms of cancer. Normally this tight regulation is achieved through the coordinated functioning of multiple signal cascades that translate signals received at the cell surface to changes in gene expression in the nucleus. These biochemical signaling pathways play central regulatory roles in a variety of intracellular functions and identification of their relevant components (e.g., proteins involved in intracellular signaling) is critical to understanding their mechanism of action.

Numerous techniques for isolating and identifying protein components of intracellular signaling pathways have evolved over the past years. Expression cloning techniques for identifying and isolating nucleic acids that encode proteins having specified biochemical activities are particularly powerful. These techniques allow the cloning and identification of genes based solely on the biochemical activities and properties of their protein products. For example, U.S. Pat. No. 4,675,285, discloses a method of expression screening large pools of cDNA clones which are transiently expressed via a mammalian expression vector in mammalian cells such as the African green monkey kidney COS cell line. However, the success of such an approach depends on the ability to detect the activity of the desired protein (as expressed from the transient expression system used) over the background signal of the endogenous proteins present in the mammalian host cells. Depending on the yield of protein from the expression system and on the sensitivity of the detection or assay system, a common problem is that any activity due to the exogenously expressed proteins is masked by the detection of a large amount of activity from the host cells, thus making it extremely difficult to detect the desired protein.

U.S. Pat. No. 5,654,150 also describes an expression cloning method. This method uses small pools of cDNA clones and in vitro transcription/translation techniques to express proteins encoded by the clones. Again, however, for many applications (especially for detecting specific enzymatic activities), the background signal from the cellular lysate used in the in vitro transcription/translation technique masks signals from the relatively low levels of proteins generated from the clones by this method. In addition, the in vitro transcription/translation technique does not permit the identification of any activity which requires an intact cell. Thus identification of activities that require or detect specific post-translational modification of proteins in mammalian cells or that require an intracellular environment (e.g., an intermediate protein or cofactor) would not be possible by this approach.

Thus, the presently available expression cloning methods are insufficient to identify and isolate many components of intracellular signaling pathways that are critical for understanding various cellular processes.

### SUMMARY OF THE INVENTION

The present invention relates to a method of mammalian expression cloning wherein a cDNA construct expresses a tagged polypeptide having a biochemical activity of interest.

More specifically, the present invention relates to a method of expression cloning wherein a mammalian expression library of cDNA constructs expressing tagged polypeptides is screened for a biochemical activity of interest. The inclusion of a specific peptide tag at the end of each protein produced by a cDNA expression library allows isolation of the expressed fusion-proteins away from the expression system's background of endogenous proteins. In addition, the appropriate choice of a mammalian expression vector and mammalian host cells allows production of adequate amounts of a mammalian (and hence correctly post-translationally modified) source of expressed proteins suitable for a screen for the biochemical activity of interest, including activities requiring intact cells.

The method comprises the steps of: a) preparing a tagged cDNA expression library comprising bacterial cells comprising (e.g., containing) tagged cDNA plasmid constructs; b) culturing the bacterial cells of step a) to produce clones where each clone corresponds to a single tagged cDNA construct; c) arraying the individual bacterial clones; d) pooling a predetermined number of arrayed clones and isolating plasmid DNA from them; e) transiently transfecting suitable mammalian host cells with the pooled plasmid clones and maintaining the transfected cells under conditions suitable for the expression of the tagged cDNA construct, thereby producing tagged polypeptides; f) assaying the expressed tagged polypeptides for a biochemical activity of interest wherein the assay involves isolating or detecting the tagged polypeptides; and identifying a pool of clones comprising a cDNA construct encoding the tagged polypeptide having the biochemical activity of interest.

The method further includes repeating steps d) through f) until a single cDNA construct expressing a tagged polypeptide having the biochemical activity of interest is identified.

The method further includes the preparation of the tagged cDNA expression library comprising the steps of: i) obtaining double-stranded cDNA from cells expressing a polypeptide with the biochemical activity of interest; ii) ligating the cDNA into an expression vector wherein the expression vector comprises a coding region for a tag operably linked to a promoter to produce a tagged cDNA construct; and iii) transforming competent bacterial cells with the tagged cDNA construct of step ii). In one embodiment, the promoter in step ii) is EF-1α and the expression vector includes sequences for the viral SV40 origin of replication. In another embodiment, the mammalian host cells in step e) are human 293T fibroblast cells expressing SV40 Large T protein which allows amplification of the transfected plasmid DNA via SV40 T mediated DNA replication. In yet another embodiment, the tag is selected from the group consisting of GST-, Myc-, HA-, FLAG- and His-.

The present invention also encompasses a cDNA construct encoding a tagged polypeptide having a biochemical activity of interest identified by the methods described herein. Expressed polypeptides identified by the methods described herein can exhibit various biochemical activities typically associated with intracellular signaling pathways. For example, the expressed polypeptide can be a substrate for a specific enzyme (e.g., protein kinase, phosphatase, etc.) involved with a cellular signaling pathway or be a specific enzyme involved in a signaling pathway. The polypeptide can interact with specific antibodies or can form specific protein-protein associations, protein-nucleic acid, protein-bio-compound associations. Alternatively, the polypeptide can be post-translationally modified, or can exhibit a particular protein or DNA association in mammalian cells in response to specific stimuli.

The method of expression cloning using a tagged cDNA library in mammalian cells, as described herein, can be used to detect any extracellular signal-regulated phenomena in intact cells. More specifically, the methods described herein can be used to study signaling cascades to further understand the process of cell control and to identify new pharmacological targets for treatment of disease where such control goes awry. In one embodiment, tagged fusion proteins expressed in host mammalian cells transfected with pools of tagged-cDNA expressing library constructs are purified away from the host cell proteins by virtue of their peptide-tags before being assayed for a biochemical activity of interest. In another embodiment, the use of the mammalian expression system of the current invention allows for a screen that detects phenomena that occur in intact cells. In one embodiment, the mammalian expression system can be used for detecting a polypeptide-protein association that occurs in vivo, and is therefore more physiologically significant. The cloning system can also be used to detect polypeptides that can only be detected when tested in vivo because the association searched for requires an intermediate protein present in the cell. In another embodiment, the mammalian transient transfection system of the current invention can be used for detecting tagged polypeptides that are modified in the cell (e.g., phosphorylated on tyrosines, glycosylated, proteolytically cleaved, etc.) in response to a specific extracellular signal such as a growth factor. This application could be valid in a variety of cell types and the effect of several biochemical stimuli can be screened. In all cases, the peptide tag on each expressed protein is used to either isolate the protein of interest away from host cell background components or as a means to detect the expressed protein above host cell background.

The mammalian expression system described herein has advantages over bacterial or in vitro expression systems. It allows the study of interactions between proteins in their natural cellular environment, where proper folding and adequate post-translational modifications are expected to occur. The peptide tag of the fusion proteins allows selection and purification of expressed protein products by chromatography on tag-specific matrices such as a Glutathione-sepharose column for GST-tagged proteins, an anti-myc, anti-HA or anti-FLAG antibody column for Myc, HA or FLAG tags respectively, or a nickel chelate affinity column for His-tagged proteins. The method of the present invention can be used to detect cDNA library-expressed fusion-proteins that interact with a specific protein under study by virtue of antibodies against the specific tag (anti-GST, anti-myc, anti-HA or anti-FLAG antibodies) in assays such as immunoprecipitation, Western blotting or Far-Western blotting. Thus, the addition of a specific peptide tag to each protein expressed by a library of cDNA expression constructs provides several new and powerful applications of expression cloning.

### BRIEF DESCRIPTION OF THE DRAWINGS

FIG. 1 is a schematic representation of one general strategy for the mammalian expression cloning system of the current invention.

FIG. 2 is a photograph of an electrophoretic gel showing the results of testing the proposed strategy for expression cloning protein kinase substrates expressed in these ‘substrate transfections’. The electrophoretic gel depicts results of kinase assays performed with protein kinase substrates either alone (−) or in the presence of XMek3 kinase (+). Products of the kinase reactions were resolved by SDS-PAGE and detected by autoradiography.

FIG. 3 is a photograph illustrating expression from a GST-tagged cDNA expression library in 293T cells. Total cell lysates of 293T cells that were transfected with either pEBG-S203 alone or 10 pools of 96 cDNA library clones each were resolved by SDS-PAGE and immunoblotted using anti-GST antisera and ECL.

FIG. 4A-4C show the results of testing the GST-tagged library in a search for XMek3 substrates. (A) The test kinase, XMek3, was produced and purified as a GST-tagged polypeptide in 293T cells. One representative pool of 96 cDNA library clones was prepared as is (Pool) or doped with a vector expressing the test substrate, pEBG-p38, at a ratio of 1:96 (Pool+). In independent ‘substrate transfections’, test substrate pools (Pool or Pool+) were expressed in varying pool sizes (96, 384, or 960) in a mixture with other plasmid pools. GST-tagged polypeptides expressed in these ‘substrate transfections’ were isolated on beads, eluted and then used in kinase assays in vitro, either alone (−) or in the presence of XMek3 kinase (+). Products of the kinase reactions were resolved by SDS-PAGE and detected by autoradiography. (B) For each ‘substrate transfection’, equal amounts of total cell lysates (lanes “a”), proteins isolated on beads (lanes “b”), or eluted from the beads (lanes “c”) were resolved by SDS-PAGE and immunoblotted using anti-GST antisera and ECL. (C) The immunoblot shown in (B) was stripped and re-probed with an anti-p38 antibody.

FIG. 5A and 5B are photographs of electrophoretic gels showing the results of experiments to determine the catalytic activity of S203. Products of the kinase reactions were resolved by SDS-PAGE and phosphorylated proteins detected by autoradiography. (A) Coomassie blue stain of resultant gel. (B) Autoradiogram of same gel. Positions of molecular size markers (in kilodaltons) are indicated on the right.

FIG. 6A and 6B show the results of testing the GST-tagged library in a search for S203 kinase substrates. (A) The kinase, S203, was produced and purified as a GST-tagged polypeptide in 293T cells. In separate transfections, pools of 96 cDNA library clones each were also expressed and purified as GST-tagged polypeptides and then tested either alone (−) or with the kinase GST-S203 in kinase assays in vitro. Products of the kinase reactions were resolved by SDS-PAGE and visualized by autoradiography. (B) Pool #1 was broken down into subpools of 12 clones each. GST-tagged polypeptides expressed in transfections of these subpools were tested in kinase assays with GST-S203. The autoradiogram shown depicts products of kinase reactions done with parent Pool#1, or representative subpools A-D.

### DETAILED DESCRIPTION OF THE INVENTION

The cDNA expression cloning strategy of the present invention can be used widely for isolating components of intracellular biochemical signaling pathways. The present invention involves screening a mammalian expression library of tagged cDNAs for a biochemical function of interest. For example, but not limited to, screening for a substrate for an enzyme (e.g., a protein kinase) in vitro, screening for specific protein-protein associations in vivo or in vitro and isolating phosphotyrosine regulated or other post-translationally modified proteins from mammalian cells in response to specific stimuli.

A key component of the method described herein is the expression of tagged polypeptides. In the method of the present invention, an expression library encoding a specific peptide tag at the end of all cDNAs expressed leads to several key advantages. One advantage of the present method is that the expressed polypeptides are rapidly isolated from any background signal due to endogenous cellular proteins by virtue of the specific tag at the end of all polypeptides generated from the expression library. This background signal often masks any signal from a library of expressed polypeptides and thus makes a screen for a particular biochemical activity difficult. Various tags (e.g., GST-, HA-, Myc-, FLAG-, His-, etc.) can be employed in the method of the invention. Expressed tagged polypeptides are purified with specific antibodies (e.g., anti-HA, anti-Myc, anti-FLAG antibodies) or by virtue of affinity to a specific compound (e.g., purification of GST-fusion proteins on Glutathione sepharose beads or purification of His-tagged proteins on nickel-chelate columns). Thus, in one embodiment of the method of the present invention, tagged polypeptides are isolated on antibody coupled matrices, or on affinity matrices. Further, for solution based biochemical assays in vitro (such as protein kinase assays to detect protein kinases or their substrates), the tagged polypeptides can be eluted off the purification matrix and then used in the assay. The kinetics and accessibility of a solution based assay is advantageous over assays performed with tagged polypeptides bound to solid matrices (e.g., beads, plates, columns, etc.) or in situ (e.g., membrane filters).

The present method also has the advantage of tracking the library of expressed tagged polypeptides with specific antibodies to the specific tags. Antibodies are available to a number of the available tags that are used in the method of the invention and are used as a means of testing levels of expression from the library. In addition, in the present method, a primary assay in a screen can constitute the immunological tracing of the expressed tagged polypeptide. For example, tagged polypeptides expressed in the library that associate with the protein under study (either co-expressed in cells or tested for association in vitro) can be initially detected by virtue of an antibody against their tag.

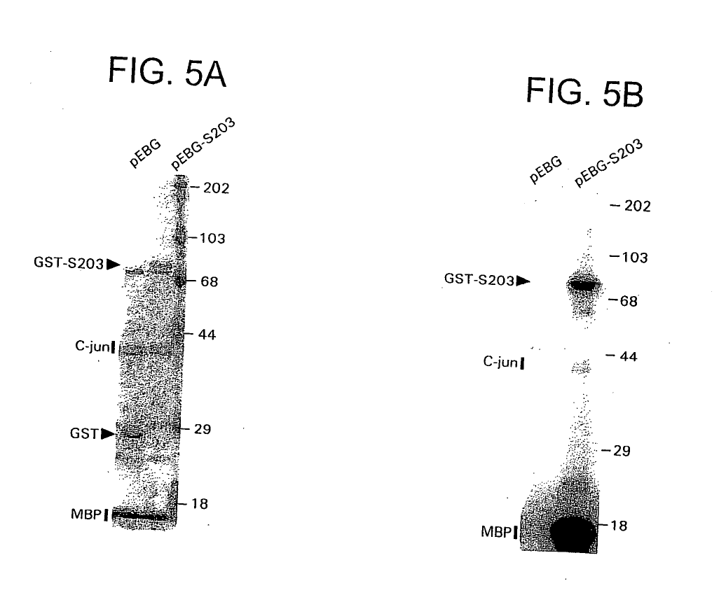

Further, in the method of the present invention, easy detection in a given assay is achieved by high levels of expression of tagged polypeptides from the library. The choice of mammalian expression vector and host mammalian cells would first be dictated by the choice of biochemical activity of interest. However in addition, a combination of expression vector and host cells that result in high levels of expression of the cDNA library constructs would be preferred. The high levels of expression of the cDNA constructs of the present invention, in addition to isolation of the expressed tagged polypeptides away from endogenous cellular background, would allow discreet and clear detection (for example, of phosphotyrosine phosphorylated proteins using an anti-phosphotyrosine antibody on Western blots). For example, high levels of expressed tagged polypeptides are obtained by the combination of the pEBG expression vector (which contains an EF-1α promoter and sequences of the SV40 origin of replication, Tanaka et al., 1995. Mol Cell Biol 15:6829-6837) and human 293T fibroblast cell transient transfections. The EF-1α promoter expresses remarkably well in 293T cells which transfect well by the calcium phosphate precipitation method. For example, as can be seen in FIG. 5A, coomassie blue detectable quantities of GST-tagged proteins were expressed transiently from the pEBG expression vector (EF-1α promoter) in 293T cells. With this combination, yields of microgram quantities of GST-purified tagged polypeptide per 10 cm tissue culture dish are routinely obtained.

The method of the present invention can be used to generate post-translationally modified tagged polypeptides from mammalian cells according to the post-translational machinery of these cells. These modifications can be responsible for regulating the functions of the tagged polypeptide and would then be useful in the detection of the biochemical activity of interest in an expression cloning system. For instance, particular modifications only present when expressed in mammalian cells, may be necessary for the association of a tagged polypeptide in the library with the co-expressed protein under study.

The method of the present invention can be used in a screen that detects a phenomenon that occurs in intact cells. Examples include detecting a protein-protein association that occurs in vivo or can only be detected when tested in vivo because it requires an intermediate protein present in the cell. A unique application of this system is detecting intracellular phenomena that are regulated by a specific stimulus received by the intact cell. For example, the current invention can be used for detecting proteins that are modified in the cell (e.g., phosphorylated on tyrosines, glycosylated proteins, etc.) in response to a specific extracellular signal such as a growth factor. Alternatively, this method could be used to detect protein-protein associations that only occur in response to a specific stimulus to an intact cell. This application is valid for a number of intracellular phenomena in a variety of cell types and the effect of several stimuli can be examined. The high levels of expression of the cDNA constructs, and the tag fused to each expressed polypeptide, allows isolation of the expressed tagged polypeptides away from endogenous cellular background and clear detection of post-translationally modified or associated expressed tagged polypeptides, for example, tyrosine phosphorylated proteins using an anti-phosphotyrosine antibody, or associated proteins using anti-tag antibodies on Western blots.

The present invention specifically relates to methods of screening a mammalian expression library of cDNA constructs where a cDNA construct expresses a tagged polypeptide that has a biochemical activity of interest. The phrase “biochemical activity of interest,” includes but is not limited to, enzyme activity, (e.g., the polypeptide is a specific enzyme, such as a protein kinase, phosphatase, acetylase, glycosylase, etc., or a substrate for a specific enzyme); protein-protein associations; protein-enzyme associations; protein-nucleic acid associations; protein-antibody associations or post-translational modifications of proteins or any of the above phenomena in mammalian cells in response to specific stimuli (e.g., phosphorylation of tyrosines, proteolytic cleavage, glycosylation, protein-protein or protein-DNA association, etc.) Therefore, the tagged polypeptide can be an enzyme, a substrate for an enzyme, a post-translationally modified protein or a protein associated with a specific antibody, nucleic acid, protein, etc.

“Solution based screening,” as used in this application, refers to any assay where the tagged polypeptides obtained by expressing the library of cDNA constructs are after purification, not bound to any solid support, for example, supports in the form of beads, fibers, filters, etc. Thus, if initial isolation of the tagged polypeptide involves the use of a solid support, they are eluted off the support before use in a solution based assay (e.g., enzymatic assay). Solution based screening has the advantage of not altering the solution kinetics of interaction between the assay components.

The term “cDNA construct,” as used in this application, refers to any vector that is introduced into a host cell. This cDNA construct may be derived from a variety of sources. These sources include genomic DNA, cDNA, synthetic DNA and combinations thereof. If the cDNA construct comprises genomic DNA, it may include naturally occurring introns, located upstream, downstream, or internal to any included genes. A cDNA construct may also include DNA derived from the same cell line or cell type as the host cell, as well as DNA which is homologous or complementary to DNA of the host cell.

The “cDNA construct” would include at least one nucleotide sequence coding for a polypeptide or protein whose production is desired, at least one nucleotide sequence coding for a tag and at least one promoter capable of regulating the expression of a resulting tagged polypeptide. In addition, signal sequences specifying secretion can be inserted into the cDNA construct. For example, the signal sequence for the mating hormone α-factor allows the efficient export of proteins into the medium. Any cDNA fragment may be useful as the starting material for the construction of cDNA constructs of the present invention. The cDNA fragment, depending on the biochemical activity of interest, could encode a enzyme, a protein, etc. A cDNA construct as contemplated by the present invention is at least capable of directing the DNA replication, and the protein expression of the nucleic acids encoding the tagged polypeptide in mammalian cells and capable of DNA replication in bacterial cells. The cDNA construct of the present invention can be derived from mammalian expression vectors and includes, for example, pcDNA1, pcDNA/Neo, pTracer™-CMV2, pCMV, pEF, pIND, pIND(SP1), pcDNA3.1, pcDNA4, pcDNA6, pEF1, pEF4, pEF6, pEBG, commercially available from various sources (for example, Invitrogen, Carlsbad, Calif., U.S.A., catalog as posted on http://www.invitrogen.com). These vectors can be modified to include a nucleic acid sequence encoding a tag operably linked to a promoter, suitable for expressing the tagged polypeptide using techniques well-known to those of skill in the art. For example, the pEBG expression vector (EF-1α promoter) allows high levels of expression of introduced genes as GST-tagged polypeptides in mammalian cells (Tanaka et al., 1995. Mol Cell Biol 15:6829-6837).

A “promoter” mediates transcription of foreign DNA sequences. A cDNA construct, as described above, may include DNA sequences required for efficient polyadenylation of the transcript, sequences of the viral SV40 origin of replication to allow SV40 large T dependent amplification of the construct in large T expressing mammalian cells and enhancers and introns with functional splice donor and acceptor sites. Promoters and enhancers consist of short arrays of DNA sequences that interact specifically with cellular proteins involved in transcription. The combination of different recognition sequences and the amounts of the cognate transcription factors determine the efficiency with which a given gene is transcribed in a particular cell type. Suitable promoters include but are not limited to, for example, the cytomegalovirus promoter, the EF-1α promoter, the SV40 early promoter, etc. In a preferred embodiment, the promoter is the EF-1α promoter.

The term “tagged polypeptides,” as used in this application, refers to a polypeptide linked to a tag, for example, His, HA, FLAG, c-Myc, GST, etc., encoded by the cDNA construct in the mammalian expression library; wherein in a cDNA construct of this invention, DNA encoding the polypeptide is linked to the DNA encoding the tag, with or without DNA encoding a cleavable linker. Thus, the attachment of the tag to the polypeptide is either cleavable or non-cleavable. The term “polypeptide” as used herein is defined as generally known to a person of ordinary skill in the art, for example, proteins, protein fragments, and synthetic polypeptides capable of being linked to a tag.

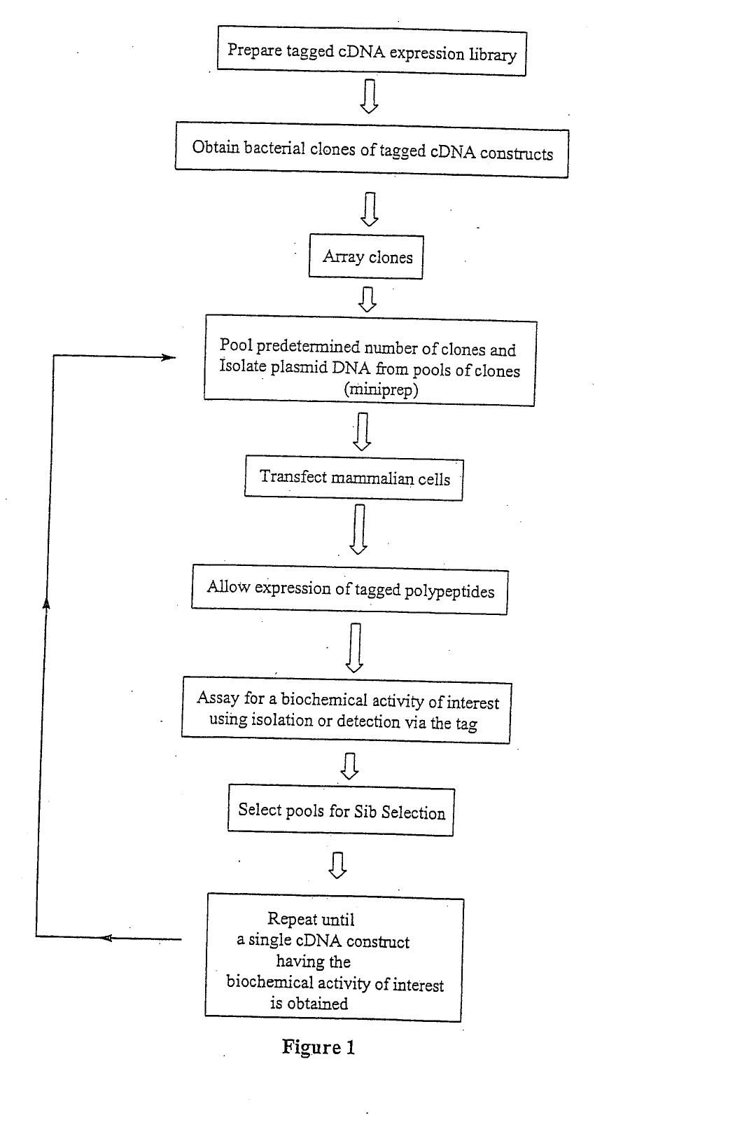

In particular, the present invention involves the following steps as shown in FIG. 1: a) preparation of tagged cDNA expression library; b) obtaining bacterial clones carrying tagged cDNA constructs; c) arraying clones; d) pooling predetermined number of clones and isolating plasmid DNA from pools of clones (miniprep); e) transfecting mammalian cells; f) allowing the expression of the tagged polypeptides; g) assaying for the biochemical activity of interest using either isolation or detection by virtue of the tag; h) selecting pools for sib selection; i) repeating steps d) through h) until a cDNA construct having the biochemical activity of interest is obtained.

Further, step a) involves the preparation of the tagged cDNA expression library by a method comprising the steps: i) obtaining double-stranded cDNA from cells expressing a polypeptide with the biochemical activity of interest; ii) ligating the cDNA into an expression vector where the expression vector comprises a coding region for a tag operably linked to a promoter to produce a tagged cDNA construct; and iii) transforming competent bacterial cells with the tagged cDNA construct of ii). A subset of cDNA constructs can be selected by an amplification method, such as PCR, to contain specific protein motifs of interest. Further, panels of cellular lysates or purified tagged proteins can be assembled from different cell types stimulated with various specific stimuli. For example, more than one expression library can be prepared and pooled where each expression library is prepared from different cell types that have been stimulated with stimuli specific for a cellular process or interaction that is to be identified.

In accordance with the present invention, any method may be used to prepare a double-stranded cDNA from a cell that expresses the desired protein, having the desired biochemical activity. Such methods are well-known to a person of skill in the art, see for example, Sambrook et al., “Molecular Cloning: A Laboratory Manual,” 2nd ED. (1989), Ausubel, F. M. et al., “Current Protocols in Molecular Biology,” (Current Protocol, 1994) and U.S. Pat. No. 5,654,150, the teachings of which are incorporated herein by reference in their entirety. There are also numerous commercially available kits for obtaining double-stranded cDNA, for example, the Superscript II™ kit (Gibco-BRL, Gaithersburg, Md., U.S.A., catalog #18248-013), the Great Lengths cDNA Synthesis Kit™ (Clontech, Palo Alto, Calif., U.S.A., catalog # K-1048-1), the cDNA Synthesis Kit (Stratagene, La Jolla, Calif., U.S.A., catalog #200301), and the like. The cDNAs may then be ligated to linker DNA sequences containing suitable restriction enzyme recognition sites. Such linker DNAs are commercially available, for example, from Promega Corporation, Madison, Wis., U.S.A. and from New England Biolabs, Beverly, Mass., U.S.A. The cDNAs may be further subjected to restriction enzyme digestion, size fractionation on columns or gels, or any other suitable method known to a person of ordinary skill in the art.

The cDNA library is then inserted into an expression vector which contains a nucleotide sequence encoding a tag, sequences that direct DNA replication in bacterial cells, and sequences that direct DNA transcription and mRNA translation in eukaryotic cells. This insertion step may optionally be performed in such a way that the cDNAs are inserted into the expression vector in a preferred direction.

Construction of suitable expression vectors is within the level of ordinary skill in the art. Many types of suitable expression vectors corresponding to the present invention are commercially available, for example, pcDNA1, pcDNA/Neo, pTracer™-CMV2, pCMV, pEF, pIND, pIND(SP1), pcDNA3.1, pcDNA4, pcDNA6, pEF1, pEF4, pEF6, pEBG etc., commercially available from various sources (see, for example, Invitrogen, Carlsbad, Calif., U.S.A., catalog as posted on http://www.invitrogen.com). These vectors can be modified to include a nucleic acid sequence encoding a tag, for example, GST-, Myc-, HA-, etc., operably linked to a promoter, for example but not limited to, EF-1α promoter, suitable for expressing the tagged polypeptide. Vectors comprising various promoters, for example, EF-1α promoter, are commercially available from many sources (for example, Invitrogen, Carlsbad, Calif., U.S.A., catalog as posted on http://www.invitrogen.com).

In the method of the present invention, following the insertion of the cDNA library into expression vectors to produce cDNA constructs, the cDNA constructs are then inserted into bacterial cells using methods such as transformation, well-known to a person of ordinary skill in the art and described in Sambrook et al., Molecular Cloning: a Laboratory Manual, 2nd Ed., Cold Spring Harbor Press (Cold Spring Harbor, N.Y., 1989). Competent bacterial cells are commercially available, for example, XL10 Gold cells are available from Stratagene Inc. The next steps of culturing bacterial cells to select for transformants and to produce individual bacterial colonies (clones) are well known in the art. Following selection of transformants on agar plates, the cultured bacterial colonies are picked individually and used to innoculate liquid culture media arranged in arrays in a grid pattern to form gridded bacterial stocks, for example, in 96-well microtiter plates. This arrangement allows representative growth of each bacterial clone in an independent well and facilitates subsequent sib-selection of positive scoring pools of clones. Following overnight growth, glycerol is added to each culture well and the bacterial stocks are stored frozen at −80° C.

In the next step of the method, a predetermined number of pools of clones are replica stamped into fresh liquid culture media and cultured to grow. Any sized pools can be made, for example, a pool of 1000 clones, 100 clones or 10 clones can be made. It is especially convenient to pool, for example, 96 bacterial colonies corresponding to the number of wells on a 96-well microtiter plate. The size of the pool is determined empirically and depends on the level of transient protein expression and the sensitivity of the detection assay for the particular biochemical activity of interest. cDNA constructs (e.g., plasmids) of the pools which comprise nucleic acid encoding the tagged polypeptides are then isolated from the pooled bacterial clones using known methods as described in Sambrook et al. Kits for performing plasmid minipreps are commercially available, for example, from Promega Corporation, Madison, Wis., U.S.A. (the Wizard Miniprep System, catalog #A7100).

After isolation of cDNA constructs by plasmid minipreps, mammalian cells are transiently transfected with the cDNA constructs and the cDNA constructs are expressed as tagged polypeptides. Transfection is a method well-known to a person of ordinary skill in the art for introducing cDNA constructs into host cells, for example, calcium phosphate- or DEAE-dextran-mediated transfection, polybrene, protoplast fusion, electroporation, liposomes, direct micro injection into nuclei, etc. Irrespective of the method used to introduce DNA into cells, the efficiency of transient transfection is determined largely by the cell type used. Suitable eukaryotic host cells are, for example, B and T lymphocytes, leukocytes, fibroblasts, hepatocytes, pancreatic cells etc. Useful mammalian cell lines would include 3T3, 3T6, STO, CHO, Ltk-, FTO2B, Hep3B, AR42J, MPC11, Cos 7, 293 fibroblast cells, etc. The frequency of transformants, and the expression level of transferred genes, will depend on the particular cell-type used and the promoter employed in the expression vector. In one embodiment of the current invention, the host cell-type is human 293T fibroblast cells and the expression vector uses the EF-1α promoter. For certain applications requiring maximum sensitivity of detection, it may be useful to label the expressed proteins with radioactive amino-acids like ³⁵S-methionine or with chemically modified amino acids like biotinylated lysine. Alternatively, the cDNA expression construct can be engineered to insert a Protein kinase A site into the fusion-proteins, thus allowing efficient labeling by in vitro phosphorylation of the purified tagged proteins by Protein kinase A and hence highly enhanced specific detection.

The expressed tagged polypeptides are then harvested from the mammalian host cells. The host cells are lysed in appropriate lysis buffers and the lysate is assayed for the biochemical activity of interest. For some applications, the tagged polypeptides are purified before being assayed. Isolation techniques used to obtain isolated tagged polypeptides include, for example, affinity chromatography, immunoprecipitation, interaction with solid support capable of binding the expressed tag of the tagged-polypeptide (in any size or form which includes, for example, beads, filter or column) or other purification techniques known in the art. For other applications, the cell lysates may be assayed directly, for example, for detection of association with a known protein, and the associated tagged protein detected by Western blotting for the tag.

The expressed tagged polypeptides are effectively maintained in a buffer solution such that they do not lose any activity being screened for in an assay for determining a biochemical activity of interest. Assays for this purpose could include, but are not limited to, detection of the protein by amido black staining, Coomassie blue staining, silver staining, fluorography, immunoprecipitation, Western blotting, autoradiography after a radioactive enzymatic assay, etc. Any suitable assay may be used in accordance with the present invention so long as the assay is capable of detecting some specific characteristic of the expressed protein, for example, immunologic, enzymatic or biochemical activity. Such assays may be based on the binding characteristics of the expressed tagged polypeptides to proteins, antibodies, nucleic acids, enzymes or any other substrate for a biochemical activity of interest. Alternatively, the effect of enzymatic activity or post-translational modification due to a biochemical stimuli on the expressed tagged polypeptide may be the basis for the assays. Representative assays are described for example, in U.S. Pat. No. 5,654,150, the teaching of which is herein incorporated by reference in its entirety.

In accordance with the present invention, the desired protein could be the substrate of a specific enzyme such as a protein kinase and could be detected in assays based on the specific kinase activity of said kinase. Pools of tagged polypeptides, as generated by transient transfection of mammalian cells as provided for in the current method, may be purified away from the endogenous proteins of the mammalian host cell by virtue of a tag-specific affinity matrix, eluted off the matrix to allow for a solution based assay in vitro, mixed with the protein kinase of interest and subjected to a protein kinase assay in vitro using radioactive γ-³²P-ATP in appropriate buffer and timing conditions. Products of the kinase assay are then resolved by sodium dodecyl sulfate-polyacrylamide gel electrophoresis (SDS-PAGE) and detected by autoradiography. For example, the ‘Exemplification’ set forth below includes examples of the detection of known and novel protein substrates of specific kinases.

In the case of protein tyrosine kinases, Western blotting with specific anti-phosphotyrosine antibodies could be used to detect tyrosine phosphorylation of potential substrates. In this case, kinase assays could be performed in vitro without the use of radioactivity. Another method would be to co-express the tyrosine kinase of interest with the pool of tagged-library constructs to detect tyrosine phosphorylation in vivo. After coexpression, the tagged proteins would be isolated away from the background of host cell proteins by virtue of their tag and then analyzed by Western blotting with specific anti-phosphotyrosine antibodies.

Alternatively, the desired protein could be the substrate of one of many other specific enzymes such as protein phosphatases, acetylases, glycosylases, ubiquitination enzymes, proteases, etc. In each case, purified and eluted tagged polypeptides, as produced according to the current method, would be subjected, in the presence of the enzyme of interest, to specific enzymatic assays which allow the detection of specific modifications in the pool of potential tagged substrate proteins. For example, the pool of tagged proteins may be, after the enzymatic reaction, resolved by SDS-PAGE and analyzed by Western-blotting with a tag-specific antibody to detect changes in their mobility on SDS-PAGE gels. In cases where there are specific antibodies available to detect the desired modification, for example anti-ubiquitin antibodies to detect ubiquitination of substrate proteins, they may be employed to probe Western blots instead. In still other cases, specific enzymatic reactions involving radioactive or fluorescent detection of substrates may be employed.

The pools of tagged polypeptides generated by the current method could be tested for the presence of specific enzymatic activities, i.e., the desired protein could be a protein kinase, phosphatase, acetylase, glycosylase, ubiquitination enzyme, protease, etc. Pools of purified tagged polypeptides could be assayed for particular enzymatic activities on test or known substrates in vitro, thus leading to the identification of novel enzymes or novel enzyme-substrate connections. Methods of detection of the enzymatic activity could involve, for example, radioactivity or fluorescence, specific antibodies such as anti-phosphotyrosine or specific anti-phosphopeptide antibodies or mobility shifts seen on SDS-PAGE analysis.

The method of the present invention allows the identification of proteins that interact specifically with a known protein of interest. Such a protein-protein interaction screen could be done in one of several ways, each employing the strengths of the present invention. The pool of tagged polypeptides may be incubated with the known protein of interest in vitro and depending on the availability of immunoprecipitating antibodies, the known protein could be immunoprecipitated and washed. Washed and immunoprecipitated complexes could be assayed by Western blotting for an associated tagged polypeptide using anti-tag antibodies. Alternatively, tagged polypeptides could be immunoprecipitated and assayed for interaction with the known protein by Western blotting using antibodies against the known protein. Instead of immunoprecipitation, the known protein could be immobilized on a resin and contacted with pools of tagged polypeptides. The resin could be washed, eluted, and protein-protein interaction could be detected by Western blotting using anti-tag antibodies. In the absence of antisera against the known protein, the interaction could also be identified by Far-Western blotting instead where cellular lysate containing the known protein could be resolved by SDS-PAGE, transferred to a membrane and then incubated with pools of tagged proteins. Associating proteins could then be detected using the anti-tag antibodies.

One powerful way to detect protein-protein interactions using the method of the present invention would be to co-express the known protein with pools of tagged cDNA constructs in appropriate mammalian cells. This would allow protein associations to occur in vivo with the correct post-translational modifications of both interacting proteins and in the presence of possible necessary cofactors or intermediate proteins. The interaction could be detected by co-immunoprecipitating the known protein with the tagged polypeptides and detecting the desired interacting protein by using anti-tag antibodies.

The method of the current invention can be used to detect polypeptides that interact with specific nucleic acid sequences. Thus, transcription factors, chromatin remodeling proteins, proteins involved in DNA replication, RNA binding proteins, etc. can be identified using the tagged polypeptides of the current invention. The specific RNA or DNA sequence could be immobilized on a solid support and incubated with pools of tagged proteins under appropriate binding conditions and bound proteins detected by SDS-PAGE followed by immunoblotting with anti-tag antibodies. Alternatively, Electrophoretic Mobility Shift Assays (EMSA or ‘DNA gel shift’) assays could be performed using specific DNA/RNA probes.

If the desired protein is specifically associated with any biological compound or element of interest, it can be detected using the method of the invention. Thus, affinity matrices of any compound/element of interest can be used in binding assays with pools of tagged polypeptides and associated polypeptides detected by SDS-PAGE followed by immunoblotting with anti-tag antibodies. Examples include compounds such as vitamins, phosphotidyl inositols, metals, etc. The high level of expression of the tagged proteins in the present invention and the ease of detecting the tagged proteins with anti-tag antibodies provide a powerful and convenient method of screening for associated proteins.

In accordance with the present invention, purified tagged polypeptides could be screened for possessing a specific biological activity such as the ability to promote or inhibit growth, differentiation, apoptosis, vascularization, motility, morphological alteration, etc. in responsive cells. Thus, pools of tagged polypeptides may be incubated with specific target tissue culture cells and the effect on the cells examined.

A significant advantage of the method of the current invention is the ability to screen for proteins that are involved in regulated events in mammalian cells. Thus, protein-protein associations, post-translational modifications such as tyrosine phosphorylation or glycosylation, proteolytic cleavages, etc., that occur only in response to a specific stimulus to the intact mammalian cell can be screened for directly using the current methodology. For example, mammalian cells transfected with pools of tagged cDNA constructs of the present invention could be stimulated with a specific growth factor for a specified amount of time. The transfected cells would then be lysed. Tagged polypeptides would be isolated by virtue of their tag, resolved by SDS-PAGE, and then analyzed by Western blotting with a specific anti-phosphotyrosine antibody to identify proteins that are phosphorylated on tyrosines only in response to the growth factor. This approach could be applied to a variety of intracellular phenomena.

In a larger scale application of the current invention, it would thus be possible to assemble panels of lysates or isolated tagged polypeptides from different cell types transfected with pools of tagged cDNA constructs and stimulated with various extra-cellular stimuli. Lysates or isolated tagged polypeptides from the combination of a particular cell type stimulated with a particular stimulus would then be available for analysis for a variety of biochemical activities. Alternatively, the same biochemical activity could be compared in different cell types or in response to different stimuli in the same cell type. Such an application would be a very valuable tool in providing functional genomics information in a systemized and targeted approach.

By extension of the current methodology, it would also be possible to generate sub-libraries of a particular cDNA expression library of tagged cDNA constructs which specifically comprise proteins or polypeptides containing specific motifs. For instance, since catalytic domains of protein kinases contain conserved and recognizable motifs at the DNA sequence level, it would be possible to design a PCR approach to assemble a subset of gridded cDNA library constructs that contain sequences encoding for kinase domains. Subsequently sub-panels of lysates or isolated tagged polypeptides of cells transfected with these sub-libraries could be made available for the study of, in this example, protein kinases only.

Pools of clones that test positively for the biochemical activity of interest can be subjected to sib-selection and further analysis until a single DNA construct corresponding to the biochemical activity of interest is obtained. The term “sib-selection,” as used in this application, refers to a system of dividing and sub-dividing a large cDNA library into a manageable number of pools, each pool consisting of between about 2 to about 1000 clones. These pools are then tested for the biochemical activity of interest. After a pool is identified that scores positively, it is subdivided into successively smaller pools, each of which is retested until the single cDNA construct of interest is isolated. By assigning individual clones to sub-pools in a matrix format, sib-selection and analysis can be performed more rapidly.

The optimal pool size for expression can be determined empirically. For example, the pool size can be small to allow for increased sensitivity and easier sib-selection. However, it would be possible to assay more clones in a given amount of time if the pool size were larger. This is particularly useful if, for example, in the mammalian expression library a majority of cDNA constructs encode out of frame tagged polypeptides. However, larger sized pools pose a problem of resolution of potential positive signals on SDS-PAGE gels, affinity columns, etc. In order to screen larger numbers of transfectants smaller (96) sized pools can be transfected into smaller-sized (35 mm) dishes in a 6-well format. For a feasible scale of sib-selection rounds, about 5-50%, more preferably about 10%, of the pools should score positively. If a higher rate of positive-scoring pools is observed, an additional filter could be added to the screen (for example, another test for the specificity of the biochemical activity of interest), before proceeding to sib-selection.

cDNA inserts of single cDNA constructs that reproducibly score positive in a screen for a biochemical activity of interest may be sequenced directly. Sequence information is expected to provide a first guide in dividing positive clones into groups of varying priority. Sequence information and homology searches can identify positive clones as known proteins or un known proteins with recognizable signaling motifs. Tagged polypeptides identified by the methods described herein that appear likely to have a signaling function are selected to follow up first.

This invention is illustrated further by the following exemplification which is not to be construed as limiting in any way.

### EXEMPLIFICATION

Expression Cloning of Substrates of Protein Kinases

Many of the known intracellular signal transduction pathways involve the regulated functioning of protein kinases. To understand the mechanism of action of such pathways, it is necessary to know the physiological substrates of these kinases. The method of the present invention can serve as a general strategy which allows solution based phosphorylation screening of proteins expressed in mammalian cells. This procedure permits direct identification of polypeptides that are substrates for a protein kinase in an assay conducted under conditions of solution kinetics with appropriate soluble amounts of mammalian expressed, and hence modified, proteins.

Description of the Method

A cDNA expression library using the pEBG expression vector is used to express GST-tagged polypeptides using the EF-1α promoter. The library clones are arrayed in a gridded pattern as bacterial stocks. A set number of cDNA constructs are isolated from their corresponding bacterial stocks and then expressed by transient transfection of 293T cells. In the next step, the expressed GST-tagged polypeptides are isolated on glutathione-sepharose beads. The isolated GST-tagged polypeptides are then eluted off the beads using excess reduced glutathione-containing elution buffer. Following elution, the eluted tagged polypeptides are used as substrates in a kinase reaction in vitro with a purified protein kinase of interest and γ-³²P-ATP. The products of the kinase reaction are then resolved by SDS-PAGE and putative kinase substrates are detected by autoradiography.

Starting with a specific sized pool of cDNA constructs and then sib-selecting positive pools down to single clones, kinase substrates are detected in a systematic and efficient manner using a mammalian source of expressed GST-tagged polypeptides in solution. Isolated in vitro substrates are then evaluated in tests for their physiological relevance.

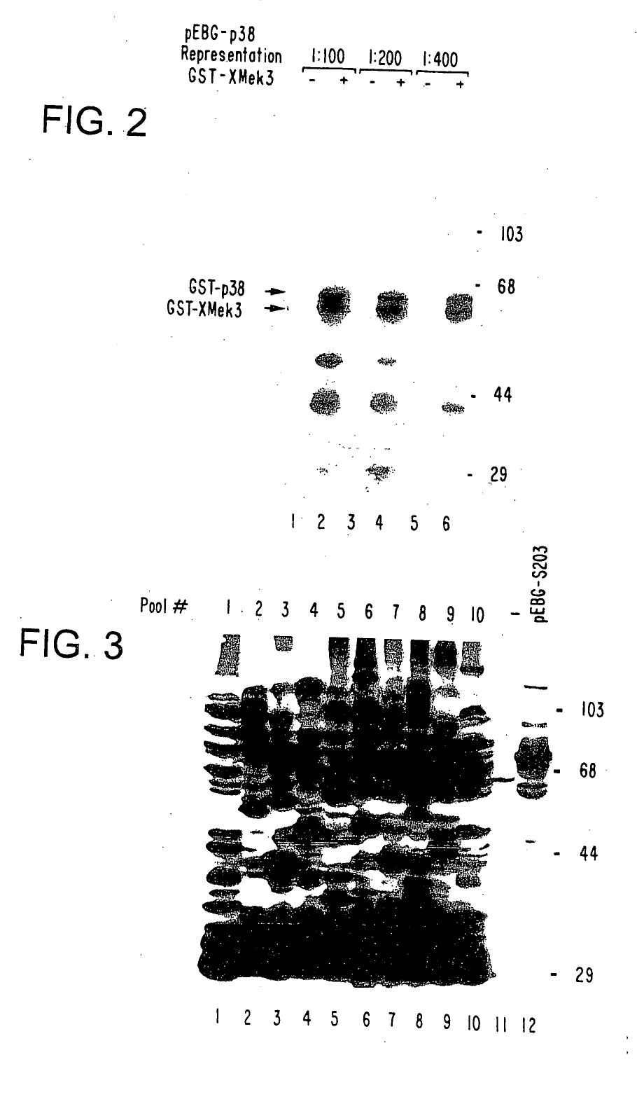

The above-described scheme was first tested using two well known protein kinase-substrate pairs belonging to the conserved mammalian map kinase signaling pathways (Marshall, C. J., 1995 Cell 80:179-185). SEK1 or XMek3 (a Xenopus homolog of MKK3) were chosen as test kinases to evaluate their ability to detect decreasingly under-represented amounts of their respective substrates, SAPK or p38, in kinase assays in vitro. The kinases, SEK1 and XMek3, were produced and purified as GST-tagged polypeptides using a pEBG vector/293T cell transfection system. In separate transfections, the substrates were expressed from the pEBG vector in varying ratios of plasmid concentration (1:1, 1:100, 1:200 or 1:400) with vector alone. GST-tagged polypeptides expressed in these ‘substrate transfections’ were isolated on beads, eluted and then used in kinase assays in vitro, either alone or in the presence of their respective kinases. As shown in FIG. 2 for XMek3/GST-p38, the substrate, GST-p38, is clearly detected in the kinase assays done in the presence of the kinase, XMek3, even at a representation level of 1:400. Identical results were obtained with SEK1/SAPK.

Construction of a GST-tagged cDNA Expression Library

Double stranded cDNA was generated from MEL cell poly (A)⁺ RNA with an oligo-dT primer and RNaseH⁻ reverse transcriptase (SuperScript II, Gibco-BRL). After adaptor ligation, the cDNA was size-fractionated (>1.2 kb) and ligated into the expression vector pEBG. A library was constructed with greater than 1.5 million primary transformants and an average cDNA insert size of 1.2 kb. Since the vector used for this library contains an N-terminal GST-moiety, the percent of clones represented in-frame ligations of cDNA to the GST-sequences was determined by testing the cDNA constructs for expression of larger than GST-sized proteins (larger than 28kD). A representative number of clones were transfected into 293T cells individually. Cell lysates were resolved by SDS-PAGE and GST-fusion proteins detected by immunoblotting with an anti-GST antibody. One of four of the clones expressed GST-tagged polypeptides of at least 40kD. Next, the expression levels of GST-tagged polypeptides, when transfected as pools of cDNA clones, were tested. In order to facilitate the organization of pools of cDNA, a portion of the expression library was plated out on agar plates as bacterial colonies and individual colonies were picked into 96 well plates to form glycerol stocks. These organized bacterial stocks could then be easily replica stamped into liquid cultures in 96 well plates and these bacterial cultures used to isolate plasmid cDNA clones in pools of 96 each. Importantly, growing each primary transformant in an independent well also allows equal representation of each transformant in the culture. FIG. 3 shows an anti-GST immunoblot of total cell lysates of 293T cells transfected with pools of 96 cDNA clones each. The large number of GST-tagged polypeptides of varying sizes detected in each lane indicates that the library yields good levels of expression and that the pEBG vector/293T cell transfection system sustains expression of high levels of each GST-tagged polypeptide even when expressed among a pool of cDNA constructs.

Testing the GST-tagged Library in a Search for Kinase Substrates

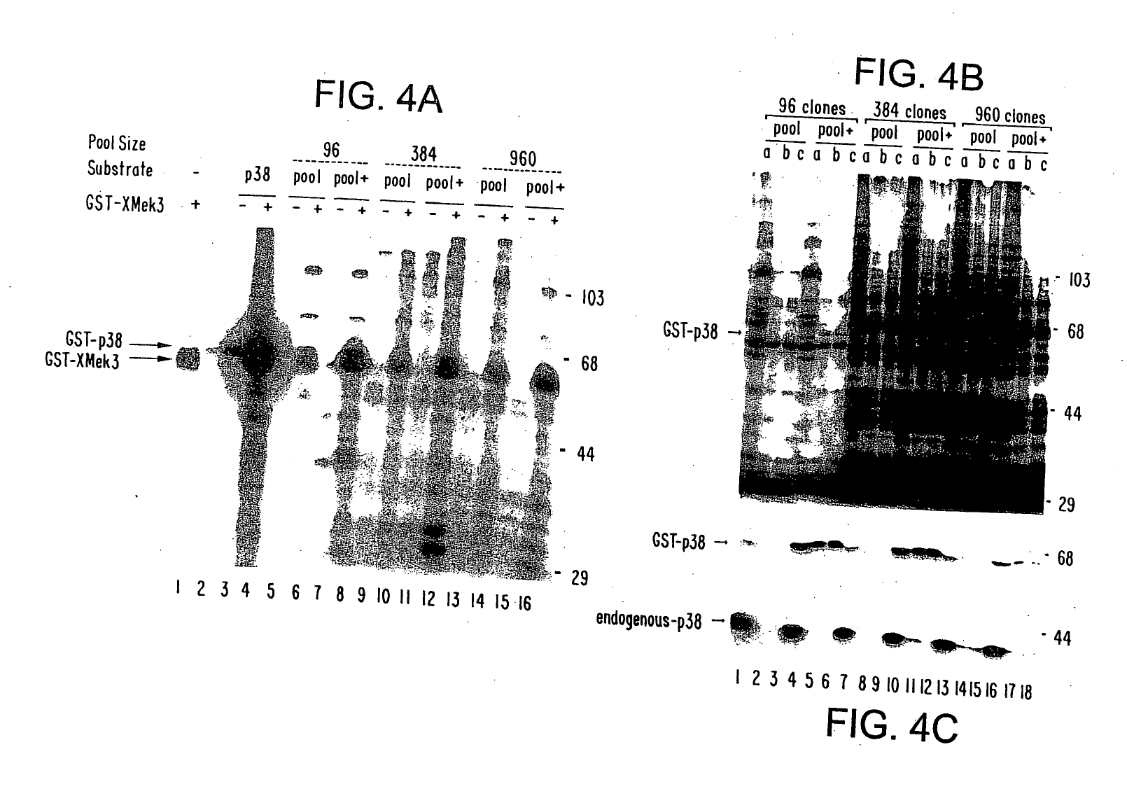

For this test, XMek3 was chosen as a test kinase and p38 as the test substrate to be searched for. One of the arrayed 96 well bacterial stock plates (Pool 10) was duplicated with one single well substituted for a pEBG-p38 transformed bacterial culture, thus creating a 96-clone sized ‘p38-doped’ pool (Pool+). Plasmid DNA was purified from both the parent Pool and Pool+. The XMek3 kinase was produced and purified as a GST-tagged polypeptide in 293T cells. In separate transfections, the candidate substrate pools (‘Pool’ or ‘Pool+’) were expressed in varying pool sizes of 96, 384 or 960 in a mixture with other plasmid pools. GST-tagged polypeptides expressed in these ‘substrate transfections’ were isolated on beads, eluted and then used in kinase assays in vitro either alone or in the presence of XMek3. As shown in FIG. 4A, in the p38-doped samples, a band corresponding to the size of GST-p38 was clearly detected in the kinase assays done in the presence of XMek3, even at a pool size of 384. In order to confirm the identity of this band and to examine the profile of GST-tagged polypeptides expressed at these pool sizes, GST-tagged polypeptide mixtures in the different pools used in the kinase assay were identified in total cell lysate, GST-tagged polypeptides isolated on beads (pull downs) or GST-tagged polypeptides eluted from the beads (elutions) by immunoblotting with an anti-GST antibody (FIG. 4B). The same blot was then stripped and probed with an anti-p38 antibody (FIG. 4C). It is clear from FIG. 4B that the expression of the GST-tagged polypeptides (total lysates), their isolation on glutathione beads (pull downs) and elutions work quite efficiently in pools of 96 and 384; pool sizes of 960 appear to not be enriched proportionally over pools of 384 and are likely over the limit of saturation of the expression system. FIG. 5C confirms that GST-p38 is expressed and purified efficiently even when in pools of 960 clones.

Testing the GST Library in Search for Substrates of a Ste20-like MST Kinase, S203

Ste20 is a critical upstream serine/threonine kinase in the conserved map kinase cascade that regulates the pheromone response in yeast (Herskowitz, I. 1995. Cell 80:199-211). Several homologs of Ste20 have been identified in mammalian cells including a sub-family of kinases, referred to as the MST kinase family, that have not been linked to any of the known mammalian map kinase pathways, and hence await identification of their substrates and assignation to a biological role (Sells, M. A. and Chemoff, J., 1997. Trends in Cell Biol 7: 162-167). S203 is a novel murine MST kinase with potent specific kinase activity.

An example of a kinase assay of S203 activity is shown in FIGS. 5A and 5B. cDNA encoding S203 was subcloned into the mammalian expression vector pEBG in order to express it as a GST-tagged polypeptide. The pEBG expression vector (EF-1α promoter) allows high levels of expression of introduced genes as GST-tagged polypeptides in mammalian cells. pEBG vector alone or the resultant plasmid, pEBG-S203, were transiently transfected into human 293T fibroblast cells using the Calcium phosphate-precipitation method. 48 hours post-transfection, cell extracts were prepared, and expressed GST-tagged polypeptides were immobilized on glutathione-agarose beads. The bound GST-tagged polypeptides were subjected to kinase assays performed in vitro with Myelin Basic Protein (MBP) or bacterially produced and purified c-jun added as substrates. Products of the kinase reactions were resolved by SDS-PAGE and phosphorylation of MBP/c-jun detected by autoradiography. As seen in the coomassie stained polyacrylamide gel depicted in FIG 5A, GST-S203 is expressed as a tagged polypeptide of about 80 kilodaltons. In addition, as shown in the autoradiogram in FIG. 5B, this 80 kD protein is able to phosphorylate itself as well as added MBP. However, c-jun appears to be a poor substrate for this active kinase.

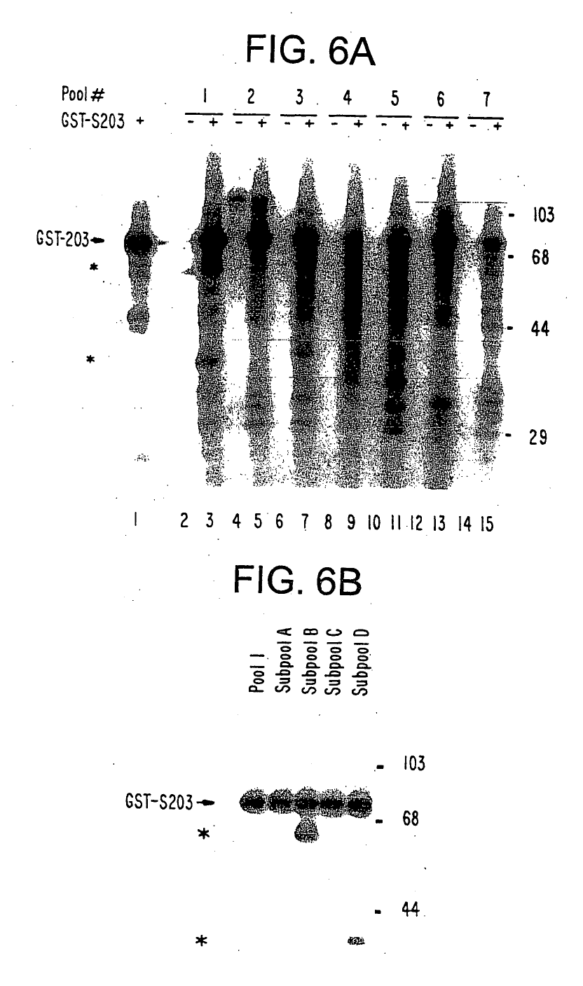

In order to examine the background and noise levels when using S203 as the kinase in a search for specific substrates among the GST-library, 24 pools of 96 clones each were tested in the strategy outlined above. Two pools yielded signals that were detectable over background and are being sib-selected down. FIG. 6A, depicts the initial screen with pools 1-7. When assayed alone, it is clear that the GST-pools themselves do not have much background kinase activity (lanes without added GST-S203). The isolated GST-S203 displays strong autokinase and some background signal. However, when GST-S203 is included in an assay with a pool containing putative substrates (Pool 1), additional signals (indicated with *) are detected. In addition, not every pool assayed displays strong signals over background. FIG. 6B shows that the signals obtained with Pool 1 are reproducible and are being sib-selected down into smaller sized pools, thus allowing their identification as single clones.

Using the method of the present invention, about 20,000 clones of the GST library have been screened and 13 individual clones sib-selected down to single constructs and sequenced. Of these, 4 represent previously unknown proteins and 9 represent known proteins that are substrates of S203 kinase in vitro. One of the known proteins identified encodes the protein kinase Polo-Like Kinase 1 (PLK1). PLK1 is a serine/threonine protein kinase implicated in the regulation of multiple aspects of cell-division and proliferation including entry and exit from M-phase, mitotic spindle assembly and cytokinesis (reviewed in Glover et al., 1998. Genes Dev 12:3777-3787). The MST kinase S203 phosphorylates and activates PLK1. Thus, the expression strategy developed and described herein has yielded the identification of a physiological relevant substrate for the MST kinase S203 and indicated, for the first time, a biological role for the family of MST kinases.

1. A method of identifying a cDNA construct wherein the cDNA construct expresses a tagged polypeptide having a biochemical activity of interest comprising the steps of:
a) preparing a tagged cDNA expression library comprising more than one tagged cDNA plasmid construct, wherein the constructs are contained in bacterial cells;
b) culturing the bacterial cells of step a) to produce clones wherein each clone corresponds to a single tagged cDNA construct;
c) arraying the individual bacterial clones;
d) pooling a predetermined number of arrayed clones and isolating plasmid DNA from them, thereby producing pooled plasmid clones;
e) transfecting suitable mammalian host cells with the pooled plasmid clones and maintaining the transfected cells under conditions suitable for the expression of the tagged cDNA construct, thereby producing tagged polypeptides;
f) assaying the expressed tagged polypeptides for a biochemical activity of interest; and
g) repeating steps d) through f) one or more times, thereby identifying a cDNA construct encoding the tagged polypeptide having the biochemical activity of interest.

2. The method of claim 1 wherein steps d) through f) are repeated until a single cDNA construct expressing a tagged polypeptide having the biochemical activity of interest is identified.

3. The method of claim 1 wherein the tagged cDNA plasmid constructs comprise a tag that is selected from the group consisting of: Glutathione S-Transferase (GST-), c-Myc (Myc-), HA-, FLAG epitope (FLAG-) and poly-Histidine (His-).

4. The method of claim 1 wherein preparing the tagged cDNA expression library of step a) comprises the steps of:
i) obtaining double-stranded cDNA from cells expressing a polypeptide with the biochemical activity of interest;
ii) ligating the cDNA into an expression vector wherein the expression vector comprises a coding region for a tag operably linked to a promoter to produce a tagged cDNA construct; and
iii) transforming competent bacterial cells with the tagged cDNA construct of step ii).

5. The method of claim 4 wherein the tagged cDNA library comprises cDNA constructs having specific protein motifs that have been selected by polymerase chain reaction.

6. The method of claim 4 wherein the promoter in step ii) is EF-1α.

7. The method of claim 1 wherein the mammalian host cells used in step e) are 293 T fibroblast cells.

8. The method of claim 1 wherein the biochemical activity of interest is selected from the group consisting of:
a) acting as a substrate for a specific enzyme;
b) being a specific enzyme;
c) interacting with specific antibodies;
d) forming specific protein-protein associations;
e) forming specific protein-nucleic acid associations;
f) interacting specifically with any biological element or compound;
g) possessing cell biological activity selected from the group consisting of: growth, differentiation, apoptosis, vascularization, motility or morphological change promoting or inhibiting;
h) undergoing specific post-translational modifications in mammalian cells;
i) possessing any of the activities in a-h only in response to a specific stimulus in mammalian cells.

9. The method of claim 1 wherein step d) each pool of clones comprises from about 2 to about 1000 clones.

10. The method of claim 1 wherein more than one expression libraries are prepared and each expression library comprises a different cell type that is stimulated with a specific stimulus.
